(19) United States
(12) Patent Application Publication
(10) Pub. No.: US 2023/0290508 A1
(43) Pub. Date: Sep. 14, 2023

(54) METHOD AND SYSTEM OF PROVIDING INFORMATION TO PREDICTING BREAST RECONSTRUCTION SURGERY PROGNOSIS

(71) Applicant: SAMSUNG LIFE PUBLIC WELFARE FOUNDATION, Seoul (KR)

(72) Inventor: Kyeong Tae LEE, Seoul (KR)

(21) Appl. No.: 18/105,516

(22) Filed: Feb. 3, 2023

(30) Foreign Application Priority Data
Feb. 7, 2022 (KR) 10-2022-0015818

(51) Int. Cl. G16H 50/20; A61B 5/00; G16H 50/30

(52) U.S. Cl. CPC G16H 50/20; A61B 5/7275; G16H 50/30

(57) ABSTRACT

A method of providing information to predict the prognosis of breast reconstruction surgery and a system for predicting the prognosis of breast reconstruction surgery is provided. By using a method or system for providing information to predict the prognosis of breast reconstruction surgery according to one specific example, it is possible to predict the occurrence of complications in a subject into which a tissue expander is inserted or scheduled to be inserted during a process of breast reconstruction surgery and to prepare appropriate treatment for the subject, and it is possible to provide information to aid in selecting a type of tissue expander with a low possibility of complication occurrence.

### FIELD OF THE INVENTION

The present invention relates to a method and system for providing information to predict the prognosis of breast reconstruction surgery.

### BACKGROUND OF THE INVENTION

Breast cancer is the most common cancer in women and is the second most fatal cancer. In 2001, the prevalence of breast cancer was 90 to 100 per 100,000 in the United States and 50 to 70 per 100,000 in Europe. The incidence of this disease is gradually increasing worldwide. Risk factors for breast cancer include race, age, mutations in tumor suppressor genes BRCA1, BRCA2, and p53, etc. Alcohol intake, high-fat diet, lack of exercise, exogenous postmenopausal hormones, and ionizing radiation also increase the risk of breast cancer. Estrogen receptor (ER-) and progesterone receptor (PR-) negative breast cancer, a large tumor size, a high-grade cytology result, and an age under 35 are associated with a poor prognosis (Goldhirsch et al. (2001). J. Clin. Oncol. 19: 3817-27).

The most essential treatment for breast cancer is a surgical operation to remove a tumor, and adjuvant therapy, such as prior chemotherapy using drugs before surgery, radiotherapy to prevent cancer recurrence after surgery, anticancer chemotherapy, anticancer hormone therapy, or the like, is performed depending on the state of the tumor. As for the adjuvant therapy, the type and order of therapy are determined according to the patient's age, presence or absence of menopause, microscopic characteristics of breast cancer, degree of metastasis, patient's symptoms, and other health conditions.

In breast cancer surgery, when cancer is present locally, the breast may be preserved by removing only the cancer and part of the normal tissue around the cancer, and when cancer has invaded lymph nodes or other tissue, the breast is completely removed and then breast reconstruction surgery may be performed to restore the breast. Reconstruction surgery after a mastectomy is divided into immediate reconstruction surgery, which is performed simultaneously with a mastectomy, and delayed reconstruction surgery, which is performed after a sufficient period of time has elapsed after surgery, depending on the reconstruction period. As the reconstruction surgery after a mastectomy, there are insertion surgery in which an implant or a tissue expander and an implant are implanted, and skin flap surgery in which autologous tissue such as skin, muscle, fat, and the like of the back or abdomen is used. The timing and method of such reconstruction surgery are determined in consideration of the patient's condition, mastectomy method, stage of breast cancer, presence or absence of metastasis, presence or absence of adjuvant therapy, and the like. Breast cancer patients who have undergone mastectomies experience not only physical changes but also psychological side effects such as low self-esteem, depression, and the like due to loss of femininity. In the case of patients who have undergone reconstruction surgery to restore the breast at the same time as a mastectomy, immediate reconstruction surgery is preferred because it has been shown to provide significant benefits to the patients in both psychological aspects and overall quality of life after treatment.

Among immediate reconstruction surgery, two-stage breast reconstruction surgery is performed after a total mastectomy, in which stage 1 surgery involves inserting a tissue expander and stage 2 surgery involves removing the tissue expander and implanting an implant. The tissue expander is expanded between the stage 1 surgery and the stage 2 surgery to minimize side effects related to the implant implanted in the stage 2 surgery.

Tissue expanders are divided into micro-textured tissue expanders and macro-textured tissue expanders depending on the surface texture that appears differently due to differences in production techniques. Macro-textured tissue expanders are produced using “lost salt technique,” in which a surface of the macro-textured tissue expander is pressed with a fine layer of salt, and have a large surface area. On the other hand, micro-textured tissue expanders are produced by engraving a surface of the micro-textured tissue expander with textured foam and have a small surface area. The surface of the micro-textured tissue expander is smoother than the surface of the macro-textured tissue expander. It is estimated that a macro-textured implant reduces the risk of malposition and rotation of the implant due to strong adhesion with the surrounding shell as compared to a micro textured tissue expander. However, when compared to micro-textured tissue expanders, macro-textured tissue expanders are more likely to form double shells or biofilms that can lead to chronic inflammation. Because of the above point, there may be the risk of developing a delayed infection, a seroma, or breast implant associated-anaplastic large cell lymphoma (BIA-ALCL).

Therefore, concerns may be raised that the type of tissue expander inserted in stage 1 surgery may affect the occurrence of complications after breast reconstruction surgery, and thus there is a need for a study on this.

### SUMMARY OF THE INVENTION

The present invention is directed to providing a method of providing information to predict the prognosis of a patient who has undergone breast reconstruction surgery after a mastectomy.

The present invention is also directed to providing a system for predicting the prognosis of a patient who has undergone breast reconstruction surgery after a mastectomy.

According to an aspect of the present invention, there is provided a method of providing information to predict a prognosis of breast reconstruction surgery, comprising collecting clinical data, calculating a pattern including the same result by classifying the clinical data on the basis of types of tissue expanders and treatment results, and predicting possibility of complication occurrence in a subject by applying the type of tissue expander among clinical data of the subject to the calculated pattern.

As used in the present invention, “breast reconstruction surgery” refers to surgery performed to restore a shape of a breast after the breast is removed in order to treat breast cancer, and it is possible to restore the breast by implanting an implant or restore the breast with autologous tissue by moving some of the muscle and skin from the patient's abdomen or back, etc.

As used in the present invention, “prognosis” means determining the presence or absence of recurrence, metastasis, complications, drug reactivity, resistance, etc. of the subject before/after treatment for subjects who have not yet been diagnosed or have been diagnosed. In the present invention, “prognosis” means predicting whether the possibility of complication occurrence after breast reconstruction surgery is high using clinical data of patients who have undergone breast reconstruction surgery.

According to one specific example of the present invention, the breast reconstruction surgery may be two-stage breast reconstruction surgery comprising inserting a tissue expander, and exchanging the tissue expander for an implant.

The tissue expander is inserted after a total mastectomy in stage 1 surgery, physiological saline is gradually injected into the tissue expander for several weeks to several months to expand the tissue expander, thereby elongating the skin of the breast and forming a shape of the breast, and thus a space in which an implant will be implanted in stage 2 surgery is secured.

According to one specific example of the present invention, the tissue expander may be a micro-textured tissue expander or a macro-textured tissue expander.

Macro-textured tissue expanders are produced using “lost salt technique,” in which a surface of the macro-textured tissue expander is pressed with a fine layer of salt, and have a large surface area. On the other hand, micro-textured tissue expanders are produced by engraving a surface of the micro-textured tissue expander with textured foam and have a small surface area.

The surface of the micro-textured tissue expander is smoother than the surface of the macro-textured tissue expander. Although it is estimated that a macro-textured implant reduces the risk of malposition and rotation of the tissue expander due to strong adhesion with the surrounding shell as compared to a micro textured implant, there are no specific study results for this.

The collecting of the clinical data is a process of obtaining information necessary for prognosis prediction. The clinical data is information related to patients who have undergone breast reconstruction surgery after a mastectomy, and may be information on age, body mass index (BMI), the presence or absence of diabetes, the presence or absence of hypertension, smoking or not, and the like.

According to an embodiment of the present invention, information related to surgery for a patient who has undergone two-stage breast reconstruction surgery may be information on the type of mastectomy, weight of a resected specimen, texture (macro texture or micro texture) and size of the tissue expander, whether or not to use an acellular dermal matrix (ADM), expansion time, the type of implant (smooth implant or textured implant), and the like.

More specifically, the clinical data is obtained from a subject who has undergone two-stage breast reconstruction surgery, and may include the type of inserted tissue expander, a treatment result, and the like.

The calculating of the pattern including the same result by classifying the clinical data on the basis of the types of tissue expanders and the treatment results is a process of calculating a pattern corresponding to the treatment result according to the type of tissue expander, that is, a prognostic prediction model, on the basis of the type of tissue expander and the treatment result in the clinical data collected for predicting the prognosis of breast reconstruction surgery.

The treatment result is a result seen in a patient who has undergone two-stage breast reconstruction surgery, and includes cancer cells being completely removed and cured, or complications occurring at a surgical site or implanted implant after breast reconstruction surgery.

According to one specific example of the present invention, the treatment result may include any one complication selected from the group consisting of infections, seromas, hematomas, severe capsular contracture, implant malposition, implant exchange, and implant removal.

The infection is a sign of local or systemic inflammation associated with an inserted tissue expander, and includes a case of being treated with antibiotic treatment or surgical operation.

The seroma is the accumulation of tissue fluid output from a surrounding wound without being absorbed. The hematoma is a state in which blood is collected in one place due to bleeding in an organ or tissue.

The capsular contracture is a symptom in which a thick shell formed around the inserted tissue expander or implant gradually hardens so as to feel hard touch, which is accompanied by inflammation and the like. Capsular contracture may be divided into several stages according to the clinical judgment of a doctor according to the degree of capsular contracture. In this specification, severe capsular contracture is defined as stage three or four diagnosed by a doctor according to the Baker capsular contracture grading system.

The implant malposition includes a case of bottoming out of the implanted implant, and a case in which the implant moves or rotates from the initial implanted position.

The pattern may be used to classify and statistically analyze the type of tissue expander and the treatment result, and thus analyze the incidence rate of complications corresponding to each type of tissue expander to predict the possibility of complication occurrence.

The predicting of the possibility of complication occurrence in the subject by applying the type of tissue expander among the clinical data of the subject to the calculated pattern is a process of predicting the possibility of complication occurrence in the subject by comparing the pattern with the calculated pattern using information on the type of tissue expander among the clinical data for the subject who needs a prognosis prediction.

The subject may be a breast cancer patient who has undergone two-stage breast reconstruction surgery after a mastectomy.

The complication may be complication that occurs after two-stage immediate reconstruction surgery using a tissue expander and an implant after a mastectomy, and may be severe capsular contracture.

According to one specific example of the present invention, the complication may be severe capsular contracture or reconstruction failure.

According to one specific example of the present invention, the predicting of the possibility of complication occurrence may include predicting that the possibility of complication occurrence is high when the type of tissue expander inserted into the subject is a macro-textured type.

The subject may be a subject into which a textured implant is implanted in stage 2 surgery.

According to an embodiment of the present invention, it was confirmed that, in a case in which a tissue expander inserted into a subject who has undergone two-stage breast reconstruction surgery is a macro-textured tissue expander (11.5%), the incidence rate of severe capsular contracture was significantly high (p=0.034), compared to a case in which the tissue expander is a micro-textured tissue expander (2.7%).

In the method of providing information to predict the prognosis of breast reconstruction surgery, it is possible to predict the occurrence of complications in patients with the tissue expanders inserted during breast reconstruction surgery and to prepare appropriate treatment for the patients, and it is possible to provide a type of tissue expander that is less likely to cause complications in patients who are scheduled to have tissue expanders inserted during breast reconstruction surgery.

The method of providing information to predict the prognosis of breast reconstruction surgery described above may be implemented as a system using a computer device.

According to another aspect of the present invention, there is provided a system for predicting the prognosis of breast reconstruction surgery.

More specifically, referring to FIG. 3, a system 100 for predicting the prognosis of breast reconstruction surgery comprises an input unit 110 that receives clinical data, a pre-processing unit 120 that classifies the clinical data on the basis of types of tissue expanders and treatment results to calculate a pattern including the same result, and an analysis unit 130 that applies a type of tissue expander among clinical data of a subject to the calculated pattern to predict the possibility of complication occurrence in the subject.

Here, descriptions common to the above-described content are omitted in order to avoid excessive complexity.

The input unit 110 receives clinical data and medical records from a medical record server or the like. The clinical data may include diagnostic information on the type of tissue expander of a patient who has undergone breast reconstruction surgery after a mastectomy and the occurrence of complications after stage 2 surgery.

According to one specific example of the present invention, the breast reconstruction surgery may be two-stage breast reconstruction surgery comprising inserting a tissue expander, and exchanging the tissue expander for an implant.

The pre-processing unit 120 classifies the clinical data on the basis of types of tissue expanders and treatment results to calculate a pattern including the same result, that is, a prognostic prediction model.

According to one specific example of the present invention, the tissue expander may be a macro-textured tissue expander or a micro-textured tissue expander.

According to one specific example of the present invention, the treatment result includes any one complication selected from the group consisting of infections, seromas, hematomas, severe capsular contracture, implant malposition, implant exchange, and implant removal.

In the present invention, in order to calculate the pattern, the incidence rate of complications may be analyzed by classifying the types of tissue expanders and the treatment results.

The analysis unit 130 analyzes the pattern calculated by the pre-processing unit 120 and the information on the type of tissue expander input to the input unit 110 to predict the possibility of complication occurrence after breast reconstruction surgery in the patient into which the tissue expander is inserted or scheduled to be inserted during breast reconstruction surgery.

For example, for subjects into which a tissue expander is inserted or scheduled to be inserted, when a micro-textured tissue expander is selected, it is predicted that the possibility of complication occurrence is low, and when a macro-textured tissue expander is selected, it is predicted that the possibility of complication occurrence is high.

According to one specific example of the present invention, the complication may be severe capsular contracture.

According to one specific example of the present invention, the analysis unit 130 may predict that the possibility of complications is high when the tissue expander of the subject is a macro-textured tissue expander.

The system 100 for predicting the prognosis of breast reconstruction surgery comprising the input unit 110, the pre-processing unit 120, and the analysis unit 130 may further comprise a display unit 140 that displays a result.

According to one specific example of the present invention, the system 100 further comprises a display unit 140 that outputs a result obtained by predicting, by the analysis unit 130, the possibility of complication occurrence in the subject.

The display unit 140 may be a separate display screen or may be a certain space in a conventional computer device in which the system 100 for predicting the prognosis is stored.

Referring to FIG. 3, the system 100 for predicting the prognosis of breast reconstruction surgery may receive the type of tissue expander of the subject who wants a breast reconstruction surgery prognosis prediction, and present the possibility of complication occurrence of breast reconstruction surgery according to the input type of tissue expander.

The system 100 for predicting the prognosis of breast reconstruction surgery according to the present invention may drive one or more programs to store the programs on a recording medium or the like, and may be implemented as a device such as a computer, a server, or the like.

The programs may include code coded in various computer languages that can be executed by a processor of a computer or server. The code may include code such as a function or the like that define functions necessary for predicting the prognosis of breast reconstruction surgery, and control code capable of controlling the code.

In addition, the recording medium on which the programs are stored is a medium readable by a computer or server, for example, a read only memory (ROM), a random-access memory (RAM), a compact disc read only memory (CD-ROM), a magnetic tape, a floppy disk, an optical disk, a Universal Serial Bus (USB) memory, a secure digital (SD) card, a micro micro-secure digital (micro-SD) card, etc., but the present invention is not limited thereto.

The system of the present invention is preferably implemented as a single hardware device, or may be implemented as an embedded device accommodated in an existing hardware device or an application downloaded and installed in the form of software, as necessary.

By using a method or system for providing information to predict the prognosis of breast reconstruction surgery according to one specific example of the present invention, it is possible to predict the occurrence of complications in a subject into which a tissue expander is inserted or scheduled to be inserted during a process of breast reconstruction surgery and to prepare appropriate treatment for the subject, and it is possible to provide information to aid in selecting a type of tissue expander with a low possibility of complication occurrence.

### BRIEF DESCRIPTION OF DRAWINGS

FIG. 1A shows results of comparing the incidence rate of complications after two-stage surgery between a group using a micro-textured tissue expander.

FIG. 1B shows results of comparing the incidence rate of complications after two-stage surgery between a group using a macro-textured tissue expander according to the type of implant.

FIG. 2 is a flowchart illustrating a method of providing information to predict the prognosis of breast reconstruction surgery according to an embodiment of the present invention.

FIG. 3 is a diagram illustrating components of a system for predicting the prognosis of breast reconstruction surgery according to an embodiment of the present invention.

FIG. 4 is a diagram illustrating an example of predicting the prognosis of breast reconstruction surgery according to the type of tissue expander used in stage 1 surgery using a system for predicting the prognosis of breast reconstruction surgery according to an embodiment of the present invention.

### DETAILED DESCRIPTION OF THE INVENTION

Hereinafter, one or more specific examples will be described in more detail with reference to embodiments. However, the embodiments are only intended to describe one or more specific examples, and the scope of the present invention is not limited to these embodiments.

Hereinafter, a process for predicting the possibility of complication occurrence after breast reconstruction surgery according to the embodiments will be described.

1. Preparation for study

1-1. Population of study

The present study was approved by the Institutional Review Board of Samsung Medical Center. Information was retrospectively collected from the medical charts for patients who have completed two-stage breast reconstruction surgery, including total mastectomy, tissue expander insertion, and implant exchange, between January 2014 and December 2018. Cases in which tissue expander insertion was delayed or cases in which autologous tissue was used together with a tissue expander were excluded. The present study embodied an established system for patient safety and medical quality management in breast reconstruction surgery.

Two types of tissue expanders, i.e., the Siltex micro-textured tissue expander and the Biocell macro-textured tissue expander, were used for stage 1 tissue expander insertion surgery. During the study period, four reconstructive surgeons performed surgery and inserted micro-textured or macro-textured tissue expanders. The type of tissue expander was determined according to the preference of the attending physician in consideration of the width, size, shape, and body habitus of opposite breast of the patient, which is not operated on.

The tissue expander was cleaned with a triple antibiotic solution (a mixed solution of gentamicin, cefazolin, and povidone-iodine solution) and then inserted into the subpectoral plane. The side surfaces of the tissue expander were covered with an acellular dermal matrix (ADM) or serratus anterior muscle fascia. Whether the ADM was used was determined after discussing with the attending physician in consideration of the patients' financial status and preferences. No set algorithm was used to determine whether to use the ADM during the study period. Two drainage tubes are usually inserted, and one of the drainage tubes was positioned on the subpectoral plane and the other was positioned on the subcutaneous plane. After surgery, a light compression dressing using cotton gauze and breast support underwear were applied. The drainage tubes were removed when the amount of drainage was less than 40 ml for two consecutive days after surgery. Prophylactic antibiotics were administered until both drainage tubes were removed.

In stage 2 surgery in which a tissue expander was removed and an implant was implanted, an implant to be implanted was determined similarly to the tissue expander insertion, regardless of the type of inserted tissue expander. After the inserted tissue expander was removed, a partial capsulectomy and a circumferential capsulotomy were performed to secure a space for the implant to be implanted. The degree of resection of the shell was similar in most of the study population, but greater resection of the shell was achieved in some cases, including those with a very dense fibrous shell around the tissue expander. In this case, a suction drainage tube was inserted to prevent hematoma after surgery. The bleeding was delicately stopped, and the appropriate implant was implanted after being strongly cleaned with a triple antibiotic solution. Other management postoperative procedures were performed in the usual way. After surgery, the patient visited an outpatient clinic at intervals of 6 to 12 months to check the results of reconstruction surgery regularly.

1-2. Collection of basic information

Patient-, stage 1 surgery-, and stage 2 surgery-related variables were retrospectively collected from a database. As for the patient-related variables, information on age, body mass index (BMI), the presence or absence of comorbidities such as diabetes and hypertension etc., smoking or not, and the like were collected. Smokers were defined as patients who smoked at the time of surgery.

As for the surgery-related variables, information on the type of mastectomy, weight of a resected specimen, size and type of tissue expander, amount of expansion during surgery, whether to use an ADM, expansion time, and type (smooth implant or textured implant) and size of an implant were collected.

The expansion time is the time taken to expand the tissue expander, and was defined as the time from the first day to the last day of expansion. Information on the occurrence of complications from each surgery was also collected. The information was classified into two groups according to the stage of the surgery and a rate of postoperative complications was analyzed.

1-3. Collection of information on complications

Examples of the complications after stage 1 surgery include infections, seromas, hematomas, wound problems (resected flap necrosis, papillary necrosis, or wound dehiscence), reoperation, malposition, tissue expander removal, and implant removal. Infection was defined as a case in which there is a sign of local or systemic inflammation and was treated with oral or intravenous antibiotics or surgical intervention. Seroma was defined as the accumulation of fluid around the tissue expander after removal of the drainage tube, requiring suction or surgical intervention. Cases of infections or seromas also included cases in which an infection or seroma occurred one month after surgery. Malposition was defined as a case in which the inserted tissue expander moves from its initial position by comparing a state immediately after surgery in which the tissue expander was inserted immediately before stage 2 surgery was performed and a state in which the tissue expander was fully expanded.

The complications after stage 2 surgery include infections, seromas, hematomas, severe capsular contracture, implant malposition, implant exchange, and implant removal. Severe capsular contracture was defined as stage three or four diagnosed by an outpatient physician according to the Baker capsular contracture grading system. Implant malposition includes a case of bottoming out of the implant, and a case in which the implant moves or rotates from the initial implanted position. The analysis of results of the stage 2 surgery was conducted on cases that were followed up for at least 6 months after surgery.

Information on a drainage tube maintenance period after stage 1 surgery was also collected. Continuous or categorical analysis was performed on a drainage tube removal delay period. The drainage tube removal delay period was defined as 3/4 or more of a maintenance period of the entire population. The case in which the drainage tube maintenance period was determined to be delayed was defined as a case in which the drainage tube was maintained for 12 or more days after surgery. Since drainage tubes are usually not inserted after stage 2 surgery, no information was collected on the drainage tube maintenance period after stage 2 surgery.

1-4. Statistical analysis

For the homogeneity of basic information that can affect the results of a micro-textured tissue expander group and a macro-textured tissue expander group, propensity score matching (PSM) between the two groups was performed. Propensity scores were evaluated using logistic regression analysis for 11 patient- and surgery-related variables (independent variables such as age, BMI, smoking status, diabetes mellitus, hypertension, neoadjuvant chemotherapy, weight of resected specimen, type of mastectomy, size of tissue expander, and dependent variables such as the type of tissue expander). Based on the calculated propensity scores, the micro-textured tissue expander group and the macro-textured tissue expander group were matched at a ratio of 1:1 and postoperative incidence rate of complication were compared.

A Pearson's Chi square test or Fisher's exact test was used to analyze categorical variables, and t tests or a Mann-Whitney test was adopted to compare continuous variables. Univariate and multivariate analysis were performed using logistic regression analysis to identify independent predictive variables for outcome.

For multivariate analysis, all patient- or surgery-related variables included reconstructive surgeons, and a backward selection model was used. A value of p<0.05 was considered statistically significant. All statistical analyses were performed using SPSS version 20 (IBM Corporation, USA).

2. Results of study

2-1. Results of information collection

According to the inclusion and exclusion criteria described above, information was collected based on 1,391 cases representing 1,324 cases. The average postoperative follow-up period from a time at which tissue expander insertion was performed was 40.0 months (range: 13 to 68 months). Of these, in 1,109 cases, the Siltex micro-textured tissue expander was used, and in 282 cases, the Biocell macro-textured tissue expander was used. It was found that in a macro-textured tissue expander group, an age and BMI were significantly high, a weight of a resected specimen was higher, and a size of the inserted tissue expander was larger. Table 1 below shows results of comparing basic characteristics between a case using a micro-textured tissue expander and a case using a macro-textured tissue expander. Referring to Table 1, it was found that there was also a significant difference between the two groups in the type of mastectomy and use rate of an ADM. The average drainage tube maintenance period after tissue expander insertion in the entire population was 10.2 days, and a median value was 10 days.

TABLE 1

| | | | |
|---|---|---|---|
| | Micro- | Macro- | |
| | textured | textured | |
| | tissue | tissue | |
| | expander | expander | P |
| Variables | group (%) | group (%) | value |

Patient-related variables

| | | | |
|---|---|---|---|
| Number of cases | 1109 (79.7) | 282 (20.3) | |
| Age | 43.6 ± 7.5 | 44.7 ± 7.8 | 0.051 |
| BMI, kg/m² | 22.3 ± 2.8 | 23.2 ± 3.7 | <0.001 |
| Underweight | 103 (9.3) | 18 (6.4) | <0.001 |
| Normal | 751 (67.7) | 164 (58.2) | |
| Overweight | 227 (20.5) | 81 (28.7) | |
| Obesity | 28 (2.5) | 19 (6.7) | |
| Diabetes | 12 (1.1) | 5 (1.8) | 0.346 |
| Hypertension | 47 (4.2) | 17 (6.0) | 0.200 |
| Smokers | 23 (2.1) | 6 (2.1) | 0.955 |
| Prior radiotherapy | 32 (2.9) | 11 (3.9) | 0.379 |
| Neoadjuvant | 87 (7.8) | 30 (10.6) | 0.131 |
| chemotherapy | | | |

Surgery-based variables

| Type of mastectomy | | | | 0.048 |
|---|---|---|---|---|
| Nipple preservation | 300 (27.1) | 60 (21.3) | | |
| Skin preservation | 809 (72.9) | | 222 (78.7) | |
| Weight of resected | 376.7 ± 181.0 | 444.3 ± 261.9 | | 0.003 |
| specimen, g | | | | |
| Size of inserted | 398.6 ± 93.2 | 426.8 ± 141.4 | | 0.039 |
| tissue expander, cc | | | | |
| Amount of expansion | 128.4 ± 94.7 | 133.4 ± 105.4 | | 0.831 |
| during surgery, cc | | | | |
| Expansion rate | 0.32 ± 0.20 | 0.32 ± 0.22 | | 0.736 |
| during surgery | | | | |
| Use of ADM | 658 (59.3) | | 141 (50.0) | 0.005 |

Based on the calculated propensity scores, a total of 552 cases, 276 cases per group, were selected for matching in each group.

Table 2 shows results of comparing basic characteristics between groups after propensity score matching. Referring to Table 2 below, it was found that the two groups have similar characteristics in the analysis of all the variables.

Complications after stage 1 surgery and complications after stage 2 surgery were analyzed by matching between groups.

TABLE 2

| | | | |
|---|---|---|---|
| | Micro-textured | Macro-textured | |
| | tissue | tissue | |
| | expander group | expander group | P |
| Variables | (%) | (%) | value |

Patient-related variables

| | | | |
|---|---|---|---|
| Number of cases | 276 (50.0) | 276 (50.0) | |
| Age | 45.0 ± 8.1 | 44.7 ± 7.7 | 0.672 |
| BMI, kg/m² | 23.1 ± 3.3 | 23.0 ± 3.3 | 0.576 |
| Underweight | 15 (5.4) | 18 (6.5) | 0.862 |
| Normal | 173 (62.7) | 164 (59.4) | |
| Overweight | 74 (26.8) | 80 (29.0) | |
| Obesity | 14 (5.1) | 14 (5.1) | |
| Diabetes | 4 (1.4) | 3 (1.1) | 0.704 |
| Hypertension | 18 (6.5) | 17 (6.2) | 0.861 |
| Smokers | 5 (1.8) | 6 (2.2) | 0.761 |
| Prior radiotherapy | 13 (4.7) | 12 (4.3) | 0.838 |
| Neoadjuvant | 29 (10.5) | 28 (10.1) | 0.889 |
| chemotherapy | | | |

Surgery-related variables

| Type of mastectomy | | | |
|---|---|---|---|
| Nipple | 68 (24.6) | 59 (21.4) | |
| preservation | | | |
| Skin preservation | 208 (75.4) | 217 (78.6) | |
| Weight of resected | 434.7 ± 228.3 | 427.2 ± 225.5 | 0.547 |
| specimen, g | | | |
| Size of inserted | 420.5 ± 97.5 | 422.6 ± 140.0 | 0.477 |
| tissue expander, | | | |
| CC | | | |
| Amount of | 136.9 ± 100.8 | 132.0 ± 103.6 | 0.397 |
| expansion during | | | |
| surgery, cc | | | |
| Expansion rate | 0.32 ± 0.20 | 0.32 ± 0.22 | 0.699 |
| during surgery | | | |
| Use of ADM | 149 (54.0) | 141 (51.1) | 0.495 |
| Average value of | 40.0 | 40.0 | 0.541 |
| postoperative | | | |
| follow-up period, | | | |
| months | | | |

2-1. Analysis of complications after stage 1 surgery

As result of comparison of complication profiles, it was found that the incidence rate of seromas was significantly lower in the group in which the macro-textured tissue expander was inserted. However, it was found that the incidence rate of complications such as infections, necrosis of resected breast flap, papillary necrosis, wound dehiscence, reoperation, and tissue expander malposition and removal were similar in the two groups. Table 3 shows results of comparing complication profiles after stage 1 surgery with matching propensity scores. Referring to Table 3 below, it was found that there was no significant difference between two groups according to the type of tissue expander in the overall incidence rate of complication.

TABLE 3

| | Micro- | Macro- | |
|---|---|---|---|
| | textured | textured | |
| | tissue | tissue | |
| | expander | expander | |
| | group (%) | group (%) | |
| Number of cases | 276 (50.0) | 276 (50.0) | |
| Number of days of | 10 | 8 | <0.001 |
| drainage tube | | | |
| retention (median | | | |
| value) | | | |
| Number of days of | 96 (34.8) | 37 (13.4) | <0.001 |
| drainage tube | | | |
| removal delay | | | |
| Total | 36 (13.0) | 23 (8.3) | 0.073 |
| complications | | | |
| Infection | 6 (2.2) | 5 (1.8) | 0.761 |
| Early occurrence | 5 (1.8) | 2 (0.7) | 0.254 |
| (≤1 month) | | | |
| Delayed occurrence | 1 (0.4) | 3 (1.1) | 0.316 |
| (>1 month) | | | |
| Seroma | 11 (4.0) | 1 (0.4) | 0.004 |
| Hematoma | 6 (2.2) | 1 (0.4) | 0.057 |
| Wound problem | 22 (8.0) | 16 (5.8) | 0.313 |
| Flap necrosis | 19 (6.9) | 12 (4.3) | 0.196 |
| Nipple necrosis | 4 (1.4) | 3 (1.1) | 0.704 |
| Wound dehiscence | 6 (2.2) | 7 (2.5) | 0.779 |
| Reoperation | 20 (7.2) | 11 (4.0) | 0.096 |
| Tissue expander | 4 (1.4) | 2 (0.7) | 0.412 |
| removal | | | |
| (Explantation) | | | |
| Tissue expander | 24/246 (9.8) | 21/249 (8.4) | 0.609 |
| exchange | | | |
| (Displacement) | | | |

It was found that delayed infections occurred in 4 cases (0.7%), of which 3 cases (1.1%) occurred in the macro-textured tissue expander group and the remaining 1 case (0.4%) occurred in the micro-textured tissue expander group. It was found that in the micro-textured tissue expander group, the incidence rate of early infections occurring within one month after surgery was higher, whereas in the macro-textured tissue expander group, the incidence rate of delayed infections was higher, but there was no significant difference. Delayed seromas did not occur in either group. It was found that a drainage tube maintenance period in the macro-textured tissue expander group was significantly shorter than in the micro-textured tissue expander group (median value 8 days vs 10 days, p<0.001) and the incidence rate of the drainage tube maintenance delay in the macro-textured tissue expander group was significantly lower than in the micro-textured tissue expander group (13.4% vs 34.8%, p<0.001). In multivariate analysis, the type of tissue expander appeared to be an independent predictive variable of the occurrence of a seroma. Table 4 shows results of multivariate analysis performed on predictive factors according to each complication. Referring to Table 4 below, the macro-textured tissue expander group showed a significantly lower incidence rate of seromas after other variables were adjusted.

TABLE 4

| | | |
|---|---|---|
| | Adjusted p | |
| Complications | value | OR (95% CI) |

Seroma

| | | |
|---|---|---|
| BMI | 0.003 | 1.259 (1.081- |
| | | 1.465) |
| Hypertension | <0.001 | 15.746 (4.021- |
| | | 61.665) |
| Type of tissue expander | | |
| Micro-textured tissue | Ref | |
| expander | | |
| Macro-textured tissue | 0.012 | 0.050 (0.005- |
| expander | | 0.523) |

Drainage tube removal delay

| | | |
|---|---|---|
| Age | 0.079 | 1.030 (0.997- |
| | | 1.065) |
| Hypertension | 0.025 | 2.642 (1.133- |
| | | 6.161) |
| Type of mastectomy | | |
| Nipple preservation | Ref | |
| Skin preservation | 0.054 | 0.555 (0.305 |
| | | 1.011) |
| Size of tissue expander | <0.001 | 1.005 (1.002- |
| | | 1.007) |
| Type of tissue expander | | |
| Micro-textured tissue | Ref | |
| expander | | |
| Macro-textured tissue | <0.001 | 0.215 (0.121- |
| expander | | 0.380) |
| Amount of initial | <0.001 | 1.005 (1.002- |
| expansion | | 1.007) |
| Hematoma | | |
| Age | 0.048 | 1.113 (1.001- |
| | | 1.238) |
| Diabetes | 0.052 | 12.083 (0.983- |
| | | 148.491) |
| Weight of resected | 0.028 | 0.991 (0.982- |
| specimen | | 0.999) |
| Size of tissue expander | 0.044 | 1.012 (1.000 |
| | | 1.025) |
| Type of tissue expander | | |
| Micro-textured tissue | Ref | |
| expander | | |
| Macro-textured tissue | 0.078 | 0.122 (0.012- |
| expander | | 1.261) |
| Amount of initial | 0.056 | 0.982 (0.964- |
| expansion | | 1.000) |

Overall complications

| | | |
|---|---|---|
| Age | 0.017 | 1.045 (1.008 |
| | | 1.084) |
| Diabetes | 0.022 | 7.155 (1.334- |
| | | 38.372) |
| Type of mastectomy | | |
| Nipple preservation | Ref | |
| Skin preservation | 0.012 | 0.429 (0.222- |
| | | 0.832) |
| Weight of resected | 0.012 | 1.002 (1.000- |
| specimen | | 1.003) |
| Amount of initial | 0.001 | 1.005 (1.002- |
| expansion | | 1.007) |

It was found that a high BMI and the presence or absence of hypertension also had a significant effect on the occurrence of seromas. Similar results were found in the occurrence of the drainage tube removal delay, and it was found that the incidence rate of the drainage tube removal delay was significantly reduced in the macro-textured tissue expander group. Meanwhile, in the analysis of overall complications such as hematomas, infections, and the like, it was found that the type of tissue expander did not have a significant effect on the occurrence of complications.

2-1. Analysis of complications after stage 2 surgery

Of the 552 matched cases, 394 cases that were followed up for at least 6 months after stage 2 surgery were analyzed, and micro-textured tissue expanders were used in 195 cases and macro-textured tissue expanders were used in 199 cases. The average interval between stage 1 surgery and stage 2 surgery was 12 months (range: 4 to 40 months). The average postoperative follow-up period after stage 2 surgery was 29 months (range: 6 to 63 months). Table 5 shows results of comparing variables related to stage 2 surgery between groups.

Referring to Table 5 below, it was found that two groups had similar basic characteristics, including the use of an ADM in stage 1 surgery, tissue expander expansion time, whether adjuvant radiotherapy was administered, and whether adjuvant chemotherapy was administered.

TABLE 5

| | Micro- | Macro- | |
|---|---|---|---|
| | textured | textured | |
| | tissue | tissue | |
| | expander | expander | |
| Variables | group (%) | group (%) | p value |
| Number of cases | 195 (49.5) | 199 (50.5) | |
| Use of ADM in | 110 (56.4) | 118 (59.3) | 0.562 |
| stage 1 surgery | | | |
| Expansion time, | 5.56 | 5.60 | 0.669 |
| months | | | |
| Interval between | 11.9 | 11.7 | 0.474 |
| stage 1 surgery | | | |
| and stage 2 | | | |
| surgery, months | | | |
| Adjuvant | 40 (20.5) | 31 (15.6) | 0.203 |
| radiotherapy | | | |
| Adjuvant | 60 (30.8) | 66 (33.2) | 0.610 |
| chemotherapy | | | |
| Type of implant | | | 0.244 |
| Smooth implant | 121 (62.1) | 112 (56.3) | |
| Textured implant | 74 (37.9) | 87 (43.7) | |
| Size of implant, | 349.3 ± 114.5 | 365.9 ± 137.9 | 0.355 |
| CC | | | |
| Use of drainage | 122 (62.6) | 129 (64.8) | 0.641 |
| tube in stage 2 | | | |
| surgery | | | |

Table 6 below shows results of comparing complication profiles between groups after stage 2 surgery. Referring to Table 6 below, it was found that there was no significant difference between the two groups in the incidence rate of complications including infections, implant malposition, and implant removal. Seromas were less incidence in the macro-textured tissue expander group, but there was no statistically significant difference (p=0.079). It was found that severe capsular contracture had a higher incidence rate in the macro-textured tissue expander group (8.5% vs 4.6%), but there was no statistically significant difference (p=0.116).

TABLE 6

| | Micro- | Macro- | |
|---|---|---|---|
| | textured | textured | |
| | tissue | tissue | |
| | expander | expander | |
| | group (%) | group (%) | P value |
| Number of cases | 195 (49.5) | 199 (50.5) | |
| Overall | 21 (10.8) | 24 (12.1) | 0.687 |
| complications | | | |
| Infection | 1 (0.5) | 1 (0.5) | 0.989 |
| Seroma | 3 (1.5) | 0 | 0.079 |
| Hematoma | 2 (1.0) | 2 (1.0) | 0.984 |
| Severe capsular | 9 (4.6) | 17 (8.5) | 0.116 |
| contracture | | | |
| Implant | 6 (3.1) | 6 (4.0) | 0.613 |
| malposition | | | |
| Implant exchange | 4 (2.1) | 3 (1.5) | 0.683 |
| Implant removal | 1 (0.5) | 2 (1.0) | 0.574 |
| Postoperative | 28.2 | 28.2 | 0.492 |
| follow-up period | | | |
| after stage 2 | | | |
| surgery (months) | | | |

Table 7 below shows results of univariate and multivariate analysis performed on predictive variables of severe capsular contracture after stage 2 surgery. Referring to Table 7 below, it was found that in multivariate analysis, the type of tissue expander had no significant effect on overall complications, except for severe capsular contracture. For severe capsular contracture, the macro-textured tissue expander group showed an approximately 3-fold higher incidence rate after other variables were adjusted. It was found that the presence or absence of diabetes, whether adjuvant radiotherapy was administered, and whether drainage tubes were inserted after stage 2 surgery also did not have an effect on the occurrence of severe capsular contracture. It was found that other variables, including reconstructive surgeon, did not have a significant effect on the occurrence of severe capsular contracture.

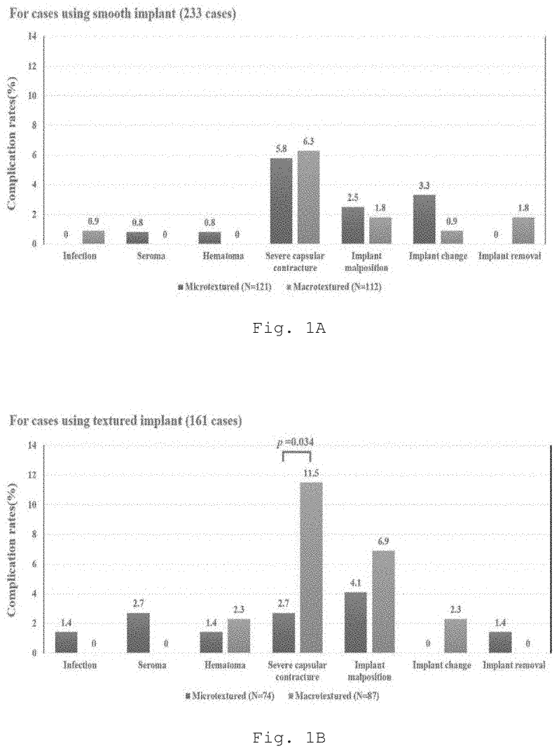

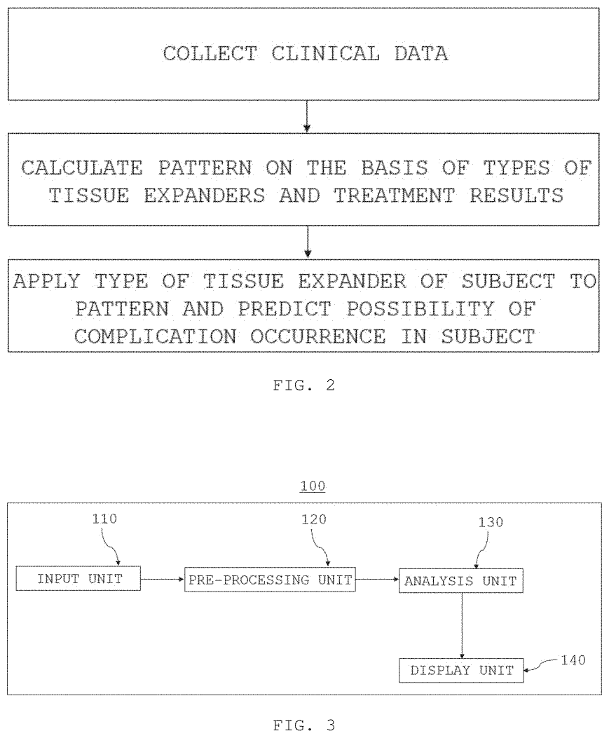

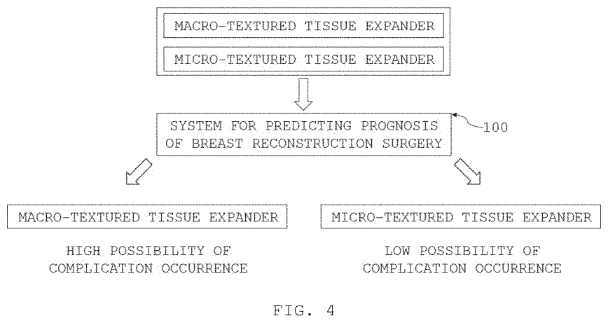

In consideration that the texture of the implant can act as a confounding factor in the occurrence of capsular contracture, additional analysis was conducted by dividing subgroups into 233 smooth implants and 161 textured implants according to the type of implant. It was found that in stage 2 surgery, the rate of drainage tube insertion was similar in groups using smooth and textured implants (63.9% vs. 63.4%, p=0.904). As a result of analyzing cases using the smooth implant, it was found that the incidence rate of complications was similar between the group using the micro-textured tissue expander and the group using the macro-textured tissue expander (see FIGS. 1A and 1B). In multivariate analysis, it was found that the type of tissue expander did not have a significant effect on the occurrence of any complication. However, in the case in which the textured implant was used, the incidence rate of severe capsular contracture was significantly higher in the macro-textured tissue expander group (11.5%) than in the micro-textured tissue expander group (2.7%, p=0.034). In multivariate analysis, it was found that the difference due to these effects was maintained, indicating that the macro-textured tissue expander had a significant effect on the occurrence of severe capsular contracture (OR, 5.078; 95% CI, 1.012-25.478; p=0.043). The incidence rate of other complications was not significantly affected by the type of tissue expander.

TABLE 7

| | Univariate analysis | | Multivariate analysis | |
|---|---|---|---|---|
| | Unadjusted | | Adjusted | OR (95% |
| Variables | p value | OR (95% CI) | p value | CI) |
| Age | 0.582 | 1.014 (0.964- | | |
| | | 1.068) | | |
| BMI | 0.760 | 0.979 (0.856- | | |
| | | 1.121) | | |
| Diabetes | 0.014 | 10.056 | 0.015 | 13.165 |
| | | (1.603 | | (1.636- |
| | | 63.079) | | 105.931) |
| Hypertension | 0.559 | 0.554 (0.071- | | |
| | | 4.182) | | |
| Smokers | 0.999 | 0 (0) | | |
| Neoadjuvant | 0.331 | 0.366 (0.048- | | |
| chemotherapy | | 2.778) | | |

Type of mastectomy

| | | |
|---|---|---|
| Skin | Ref | |
| preservation | | |
| Nipple | 0.116 | 0.5 |
| preservation | | |
| Weight of | 0.559 | 0.999 (0.997- |
| resected | | 1.001) |
| specimen | | |

Type of tissue expander

| | | | | |
|---|---|---|---|---|
| Siltex micro- | Ref | | Ref | |
| textured tissue | | | | |
| expander | | | | |
| Biocell macro | 1.109 | 1.941 (0.843- | 0.022 | 3.012 |
| textured tissue | | 4.468) | | (1.169- |
| expander | | | | 7.759) |
| Size of tissue | 0.552 | 0.999 (0.995- | | |
| expander | | 1.002) | | |
| Use of ADM | 0.451 | 1.379 (0.599 | | |
| | | 3.175) | | |
| Amount of | 0.713 | 1.001 (0.997- | | |
| initial | | 1.004) | | |
| expansion | | | | |
| Adjuvant | 0.246 | 1.615 (0.719- | | |
| chemotherapy | | 3.626) | | |
| Adjuvant | 0.005 | 3.414 (1.442- | <0.001 | 6.748 |
| radiotherapy | | 8.086) | | (2.494- |
| | | | | 18.255) |
| Expansion time | 0.122 | 0.901 (0.789- | | |
| | | 1.028) | | |
| Size of implant | 0.542 | 0.999 (0.996- | | |
| | | 1.002) | | |
| Type of implant | | | | |
| Smooth implant | Ref | | Ref | |
| Siltex tissue | 0.505 | 1.322 (0.583- | 0.403 | 1.467 |
| expander | | 2.999) | | (0.598- |
| | | | | 3.602) |
| Biocell tissue | 0.848 | 0.816 (0.102- | 0.728 | 0.676 |
| expander | | 6.545) | | (0.074- |
| | | | | 6.146) |
| Drainage tube | 0.013 | 4.708 (1.388- | 0.037 | 3.826 |
| insertion in | | 15.968) | | (1.087- |
| stage 2 surgery | | | | 13.465) |
| Infection after | 0.999 | 0 (0) | | |
| stage 2 surgery | | | | |
| Hematoma after | 0.008 | 15.250 | | |
| stage 2 surgery | | (2.058- | | |
| | | 113.022) | | |

Reconstructive surgeons

| | | |
|---|---|---|
| Doctor A | Ref | |
| Doctor B | 0.167 | 0.321 (0.064 |
| | | 1.607) |
| Doctor C | 0.833 | 0.896 (0.325- |
| | | 2.474) |
| Doctor D | 0.141 | 0.441 (0.149- |
| | | 1.310) |

In summary, according to the judgment criteria derived from the above-described embodiments, information and a system for predicting the possibility of complication occurrence after stage 2 surgery for patients undergoing two-stage breast reconstruction surgery, according to the type of tissue expander inserted in stage 1 surgery, can be provided.

While the present invention has been particularly described with reference to exemplary embodiments. It will be understood by those skilled in the art that the present invention may be implemented in modified forms without departing from the spirit and scope of the present invention. Therefore, the disclosed embodiments should be considered from an illustrative point of view for description rather than a limiting point of view. The scope of the present invention is indicated in the claims rather than in the foregoing description, and all differences within the scope equivalent thereto should be construed as being included in the present invention.

Description of Reference Numerals
100: system for predicting
110: input unit
120: pre-processing unit
130: analysis unit
140: display unit

1. A method of providing information to predict a prognosis of breast reconstruction surgery, comprising:
collecting clinical data;
calculating a pattern including the same result by classifying the clinical data on the basis of types of tissue expanders and treatment results; and
predicting possibility of complication occurrence in a subject by applying a type of tissue expander among clinical data of the subject to the calculated pattern.

2. The method of claim 1, wherein the breast reconstruction surgery is two-stage breast reconstruction surgery comprising:
inserting a tissue expander; and
exchanging the tissue expander for an implant.

3. The method of claim 1, wherein the tissue expander is a micro-textured tissue expander or a macro-textured tissue expander.

4. The method of claim 1, wherein the treatment results include any one complication selected from the group consisting of infections, seromas, hematomas, severe capsular contracture, implant malposition, implant exchange, and implant removal.

5. The method of claim 1, wherein the complication is severe capsular contracture.

6. The method of claim 1, wherein, in the predicting the possibility of complication occurrence, when the type of tissue expander used for the subject is a macro-textured tissue expander, the possibility of complication occurrence is predicted to be high.

7. A system for predicting a prognosis of breast reconstruction surgery, comprising:
an input unit configured to receive clinical data;
a pre-processing unit configured to classify the clinical data on the basis of types of tissue expanders and treatment results to calculate a pattern including the same result; and
an analysis unit configured to apply a type of tissue expander among clinical data of a subject to the calculated pattern to predict a possibility of complication occurrence in the subject.

8. The system of claim 7, wherein the breast reconstruction surgery is two-stage breast reconstruction surgery comprising:
inserting a tissue expander; and
exchanging the tissue expander for an implant.

9. The system of claim 7, wherein the tissue expander is a micro-textured tissue expander or a macro-textured tissue expander.

10. The system of claim 7, wherein the treatment results include any one complication selected from the group consisting of infections, seromas, hematomas, severe capsular contracture, implant malposition, implant exchange, and implant removal.

11. The system of claim 7, wherein the complication is severe capsular contracture.

12. The system of claim 7, wherein the analysis unit predicts that the possibility of complication occurrence is high when the type of tissue expander used for the subject is a macro-textured tissue expander.

13. The system of claim 7, further comprising a display unit configured to output a result of predicting the possibility of complication occurrence of the subject in the analysis unit.
